SEQUENCE LISTING (44 sequences)

SEQ ID NO: 1 - DNA, 15 bp, Artificial Sequence
  Probe
  misc_feature (8)..(8): n is a, c, g, or t
agtcgcgnctcagct

SEQ ID NO: 2 - DNA, 15 bp, Artificial Sequence
  Probe
  misc_feature (8)..(8): n is a, c, g, or t
agatttcnctgtagc

SEQ ID NO: 3 - DNA, 15 bp, Artificial Sequence
  Probe
  misc_feature (8)..(8): n is a, c, g, or t
tacgccancagctcc

SEQ ID NO: 4 - RNA, 15 nt, Homo sapiens
  misc_feature (8)..(8): n is a, c, g, or u
ucagcgcngagucga

SEQ ID NO: 5 - DNA, 15 bp, Homo sapiens
  misc_feature (8)..(8): n is a, c, g, or t
tcagcgcngagtcga

SEQ ID NO: 6 - DNA, 15 bp, Homo sapiens
  misc_feature (8)..(8): n is a, c, g, or t
tctaaagngacatcg

SEQ ID NO: 7 - DNA, 15 bp, Homo sapiens
  misc_feature (8)..(8): n is a, c, g, or t
ggagctgntggcgta

SEQ ID NO: 8 - DNA, 15 bp, Artificial Sequence
  Probe
tggactctctcaatg

SEQ ID NO: 9 - DNA, 15 bp, Artificial Sequence
  Probe
  misc_feature (8)..(8): n is a, c, g, or t
tggactcnctcaatg

SEQ ID NO: 10 - DNA, 15 bp, Homo sapiens
acctgagagagttac

SEQ ID NO: 11 - DNA, 15 bp, Homo sapiens
acctgagtgagttac

SEQ ID NO: 12 - DNA, 15 bp, Homo sapiens
acctgagcgagttac

SEQ ID NO: 13 - DNA, 15 bp, Homo sapiens
acctgagggagttac

SEQ ID NO: 14 - DNA, 15 bp, Homo sapiens
tctaaagagacatcg

SEQ ID NO: 15 - DNA, 15 bp, Homo sapiens
tctaaagtgacatcg

SEQ ID NO: 16 - DNA, 50 bp, Homo sapiens
tatcgtagccaatgaggtttatccgaggcgtgattattgctaattgaaaa

SEQ ID NO: 17 - DNA, 50 bp, Homo sapiens
gcactggcctcctaagccagggattgtgggttcgagtcccacctggggta

SEQ ID NO: 18 - DNA, 50 bp, Homo sapiens
aagattagcatgaggatgacacgcaaattcgtgaagcgttccatttcttt

SEQ ID NO: 19 - DNA, 50 bp, Homo sapiens
ggcccttgtgcaaggatgacacgcaaattcgtgaagcgttccatattttt

SEQ ID NO: 20 - DNA, 50 bp, Homo sapiens
ggcccctgcacagggatgacacgcaaattcgtgaagcgttccatattttt

SEQ ID NO: 21 - DNA, 50 bp, Homo sapiens
ggcccctgtgcaaggatgacacgcaaattcgtgaagcgttccatattttt

SEQ ID NO: 22 - DNA, 50 bp, Homo sapiens
ggcccctgcgcaaggatgacatgcaaattcgtgaagcgttccatattttt

SEQ ID NO: 23 - DNA, 50 bp, Homo sapiens
ggcccctgtgcaaggatgacatgcaaattcgtgaagcgttccatattttt

SEQ ID NO: 24 - DNA, 50 bp, Homo sapiens
cttctctccgtattggggagtgagagggagagaacgcggtvtgagtggtt

SEQ ID NO: 25 - DNA, 50 bp, Homo sapiens
gcgcatgcttagcatgcatgaggtcccgggttcgatccccagcatctcca

SEQ ID NO: 26 - DNA, 50 bp, Homo sapiens
gcgtctgattccggatcagaagattgagggttcgagtcccttcgtggtcg

SEQ ID NO: 27 - DNA, 50 bp, Homo sapiens
atggcccctgcgcaaggatgacacgcaaatttgtgaaggattccatattt

SEQ ID NO: 28 - DNA, 50 bp, Homo sapiens
ggcccctgtgcaagaatgactcgcaaattcgtgaagcgttccatattttt

SEQ ID NO: 29 - DNA, 50 bp, Homo sapiens
gcttcttttactctttcccttcattctcactactgtacctgattcgtctt

SEQ ID NO: 30 - DNA, 50 bp, Homo sapiens
ggcccctgcgcaaggatgacatgcaaatttgtgaagtgttccatattttt

SEQ ID NO: 31 - RNA, 50 nt, Homo sapiens
gugucaccaacguugguauacaaccccccacaacuaaauuugacuggcuu

SEQ ID NO: 32 - DNA, 50 bp, Homo sapiens
ctttttgactgtagagcaagaggtccctggttcaaatccaggttctccct

SEQ ID NO: 33 - DNA, 50 bp, Homo sapiens
tcacttcacgcatcgatctggtattgcagtacctccaggaacagtgcacc

SEQ ID NO: 34 - DNA, 50 bp, Homo sapiens
gtatcgtagccaatgaggtttatccaaggtgcgattattgctaattgaaa

SEQ ID NO: 35 - DNA, 50 bp, Homo sapiens
tgctggcggcttcccatccgctggttctatcctcaaacgccgggacaccg

SEQ ID NO: 36 - DNA, 30 bp, Homo sapiens
cattgatcatcgacacttcgaacgcacttg

SEQ ID NO: 37 - DNA, 50 bp, Homo sapiens
gcgtcagtctcataatctgaaggtcctgagttcgagcctcagagagggca

SEQ ID NO: 38 - DNA, 50 bp, Homo sapiens
gcgcctgtctagtaaacaggagatcctgggttcgaatcccagcggtgcct

SEQ ID NO: 39 - DNA, 50 bp, Homo sapiens
gcataaaacttaaaattttataatcagaggttcaactcctcttcttaaca

SEQ ID NO: 40 - DNA, 50 bp, Homo sapiens
gtctttgttgaactctttccctccttctcattactgtacttgaccagtct

SEQ ID NO: 41 - DNA, 50 bp, Homo sapiens
gctaccctggaaccttgttatgacatctgcacattacccatctgacctga

SEQ ID NO: 42 - DNA, 50 bp, Homo sapiens
gatggcatgacccctgatcaaggacggcatgcaaatttgtgaagtatttc

SEQ ID NO: 43 - DNA, 40 bp, Homo sapiens
tttcaccgccgcggcccgggttcgattcccggtcagggaa

SEQ ID NO: 44 - DNA, 40 bp, Homo sapiens
tgcacgtatgaggccccgggttcaatccccggcatctcca

(19) United States
(12) Patent Application Publication
(10) Pub. No.: US 2024/0011078 A1
(43) Pub. Date: Jan. 11, 2024

(54) SINGLE POINT VARIANT DETECTION

(71) Applicants: The University of Birmingham, Birmingham (GB); Wigner Research Centre for Physics, Budapest (HU); The University of Pécs, Pécs (HU)

(72) Inventors: John S. FOSSEY, Birmingham (GB); Miklos VERES, Budapest (HU); Huy van NGUYEN, Birmingham (GB); Roman HOLOMB, Budapest (HU); James H. R. TUCKER, Birmingham (GB); Attila SIK, Pécs (HU); Ferenc MULLER, Birmingham (GB)

(73) Assignees: The University of Birmingham, Birmingham (GB); Wigner Research Centre for Physics, Budapest (HU); The University of Pécs, Pécs (HU)

(21) Appl. No.: 18/035,055

(22) PCT Filed: Nov. 2, 2021
(86) PCT No.: PCT/GB2021/052835

§ 371 Date: May 2, 2023

(30) Foreign Application Priority Data
Nov. 2, 2020 (GB) 2017378.7
Nov. 3, 2020 (GB) 2017378.7

(51) Int. Cl. C12Q 1/6827; C12Q 1/6883; G01N 21/65

(52) U.S. Cl. CPC C12Q 1/6827; C12Q 1/6883; G01N 21/65; C12Q 2600/156

(57) ABSTRACT

The invention relates to a genetic probe, wherein the genetic probe comprises: an oligonucleotide, or an oligonucleotide analogue, with a Raman-active moiety incorporated therein, wherein the Raman-active moiety is incorporated into a base of the oligonucleotide or oligonucleotide thereof; and associated methods, uses, kits and compositions for determining a single point variant nucleotide in a target nucleic acid.

### CROSS REFERENCE TO RELATED APPLICATIONS

This application is a 35 U.S.C. § 371 National Phase Entry of International Application No. PCT/GB2021/052835 filed Nov. 2, 2021, which designates the U.S., and which claims benefit under 35 U.S.C. § 119(b) of Great Britain Application No. 2017378.7 filed on Nov. 2, 2020, the contents of each of which are incorporated herein by reference in their entireties.

### SEQUENCE LISTING

The instant application contains a Sequence Listing that has been submitted in ASCII format via Patent Center and is hereby incorporated by reference in its entirety. Said ASCII copy, created on Jan. 10, 2022, is named “JDM108949P.WOP.txt” and is 7,554 bytes in size.

### BACKGROUND TO THE INVENTION

Single point variants (that include single-nucleotide polymorphisms or SNPs) in a gene sequence are markers for a variety of human diseases, such as Alzheimer's and certain types of cancer. There is an ongoing need to detect point variants at a single locus with high specificity and sensitivity for the practical implementation of personalised medicine. The design of probes that can discriminate between the different DNA bases is an area of research that is looking to improve the diagnosis of such diseases with a genetic component.

There are several commercial or literature-based methods for detecting single point variants. These include enzyme-based approaches, and fluorescence assays. While enzyme-based methods are highly accurate, they are time-consuming and expensive. Hence, the most common methods for point variant detection involve hybridisation assays and typically the use of fluorophore-tagged DNA-based probes. The main challenges for such base-discriminating (hybridisation) systems are both lowering the limit and increasing the speed of detection.

Fluorophore-labelled assays can suffer from the inherent limitation of auto fluorescence in biological samples as well as photo bleaching. Electrochemical assays offer some advantages over the fluorophore-labelled methods, the main one being the limit of detection to as low as 100 femtomolar. It is the use of field effect transistor (FET) that has really boosted the limit of detection and offers the promise for highly sensitive DNA sensors to be on chip. Whilst there have been promising developments in this area, further increases in specificity are desired.

Therefore, an aim of the present invention is to provide improved single point variant sensing methodology, overcoming the current challenges of inherent limitation of auto fluorescence and/or background fluorescence and the provision of improved probes for such single point variant sensing.

According to a first aspect of the present invention, there is provided a genetic probe, wherein the genetic probe comprises:
an oligonucleotide, or an oligonucleotide analogue, with a Raman-active moiety incorporated therein, wherein the Raman-active moiety is incorporated into a base of the oligonucleotide, or analogue thereof.

The genetic probe may be for determining the identity of a single targeted nucleotide in a target nucleic acid. The genetic probe may be for determining the identity or presence of a nucleic acid, such as a microRNA.

Raman spectroscopy measures the light scattering caused by changes in the vibrational properties of the molecule due to bond switching during chemical reaction, conformational changes, and non-bonding (electrostatic, dipolar, hydrogen bonding etc.) interactions. Hydrogen bonding plays a crucial role during the base pair formation in nucleic acid (DNA and RNA) hybridisation. The present invention identifies a use of Raman spectroscopy to detect DNA hybridisation with high specificity. The problems of using Raman spectroscopy in this field is typically the low concentration of the molecules in a real biological situation. However, the provision of label-free molecular Raman tags and stimulated Raman spectroscopy has enabled imaging of the biological processes in real time. Furthermore, the invention uses a system that does not generate different read-outs through differences in target strand binding efficiency, rather through differences in the environment that it experiences upon duplex formation. This allows the sensing to be undertaken at any chosen fixed temperature (e.g. room or physiological temperature), and is capable of determining if a duplex is formed and whether the matched or a mismatched base is presented to the probe base.

The Raman-Active Moiety

In one embodiment, the Raman-active moiety comprises or consists of a molecule/moiety that resonates at a frequency within the cell-silent range, such as between 1800 and 2800 cm⁻¹. The Raman-active moiety may resonate at a frequency of in the approximate region of 2050 to about 2250 cm⁻¹.

The Raman-active moiety may comprise or consist of one or more of the functional groups selected from a diyne group, an alkyne group, an azide group, a nitrile/cyano group, a metal-carbonyl complex, a carbon-13 label, and a deuterium group. In one embodiment, the Raman-active moiety comprises or consists of an alkyne group. Multiple conjugated forms of these groups may be provided, for example the Raman-active moiety may comprise or consist of multiple conjugated alkynes as well as multiple instances of alkynes. The skilled person will recognise that other Raman-active moieties may be used.

The Raman-active moiety incorporated into a base or base analogue may together form a “Raman-active molecule”. In particular, the “Raman-active molecule” may comprise or consist of a nucleotide base, or an analogue thereof, comprising a Raman-active moiety, such as an alkyne group.

In one embodiment, the Raman-active molecule comprises or consists of an alkyne derivative of a nucleobase. The alkyne derivative of a nucleobase may be incorporated into the oligonucleotide sequence. Raman-active molecule may substitute a nucleobase (i.e. it may not anchor onto the oligonucleotide backbone in addition to a normal nucleobase at a selected position).

In another embodiment, the Raman-active molecule comprises or consists of a multiple conjugated alkyne derivative of a nucleobase. In another embodiment, the Raman-active molecule comprises or consists of a diyne derivative of a nucleobase. In another embodiment, the Raman-active molecule comprises or consists of an azide derivative of a nucleobase. In another embodiment, the Raman-active moiety comprises or consists of a nitrile derivative of a nucleobase. In another embodiment, the Raman-active moiety comprises or consists of a metal-carbonyl complex derivative of a nucleobase. In another embodiment, the Raman-active moiety comprises or consists of a deuterium label molecule derivative of a nucleobase. In another embodiment, the Raman-active moiety comprises or consists of a carbon-13 label derivative of a nucleobase. In another embodiment, the Raman-active moiety comprises or consists of a cyano derivative of a nucleobase. The term “derivative of a nucleobase” is intended to mean a nucleobase that has been modified to comprise a Raman-active moiety, such as those Raman-active moieties described herein.

The alkyne may be incorporated into the oligonucleotide as a moiety of 5-ethynyl-dU-CE phosphoramidite, for example according to Formula I. This includes an example of functionality permitting ready incorporation into oligonucleotide sequences by standard protocols, exemplified but not limited to the drawn phosphoramidite and DMT (N,N-dimethyltryptamine) groups.

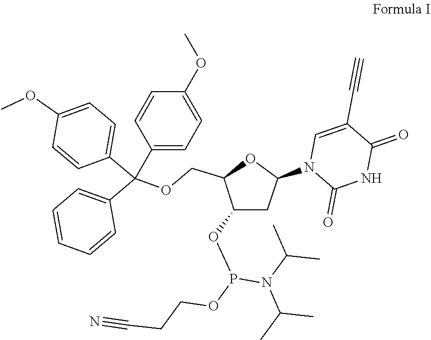 [SMILES: C#Cc1cn([C@H]2C[C@H](OP(OCCC#N)N(C(C)C)C(C)C)[C@@H](COC(c3ccccc3)(c3ccc(OC)cc3)c3ccc(OC)cc3)O2)c(=O)[nH]c1=O]

Incorporation of 5-ethynyl-dU-CE phosphoramidite into the backbone of the oligonucleotide results in the loss of the DMT group and oxidation of the phosphoramidite to phosphorous V generally seen in the backbone of DNA, for example according to Formula II.

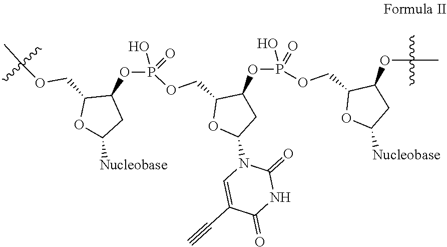

The skilled person will recognise that 5-ethynyl-dU-CE phosphoramidite provides a uracil homologue with a Raman-active alkyne moiety therein. Such a molecule would naturally base pair with adenine in a hybridisation of the genetic probe to a target nucleic acid. Attempting to base pair with an opposing thymidine, cytosine or guanine would be considered to be a mismatch. The 5-ethynyl-dU-CE phosphoramidite may substitute uracil, for example in RNA, or thymidine, for example in DNA. In one embodiment, the oligonucleotide comprises the structure provided in Formula II.

Formulas III-IX provide alternative example alkyne-comprising base analogues incorporated into the oligonucleotide as Raman-active molecules. The alkyne analogue of Formula III is derived from commercially available EdU (surrogate for thymine (T)). The alkyne analogue of Formula IV is derived from a synthetically feasible isomer of EdU (surrogate for thymine (T)). The alkyne analogue of Formula V is a synthetically feasible alkyne-containing cytosine (A) analogue. The alkyne analogue of Formula VI is a synthetically feasible alkyne-containing cytosine (C) analogue. The alkyne analogue of Formula VII is a synthetically feasible alkyne-containing cytosine (A) analogue. The alkyne analogue of Formula VIII is a synthetically feasible isomer of alkyne-containing cytosine (C) analogue. The alkyne analogue of Formula IX is a synthetically feasible alkyne-containing cytosine (G) analogue.

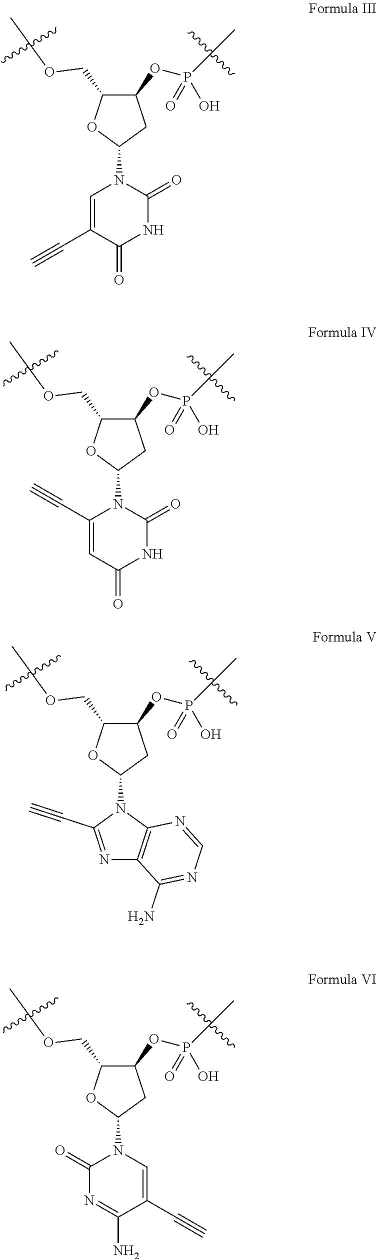 [SMILES: *OC[C@H]1O[C@@H](n2cc(C#C)c(=O)[nH]c2=O)C[C@@H]1OP(*)(=O)O ; *OC[C@H]1O[C@@H](n2c(C#C)cc(=O)[nH]c2=O)C[C@@H]1OP(*)(=O)O ; *OC[C@H]1O[C@@H](n2c(C#C)nc3c(N)ncnc32)C[C@@H]1OP(*)(=O)O ; *OC[C@H]1O[C@@H](n2cc(C#C)c(N)nc2=O)C[C@@H]1OP(*)(=O)O]

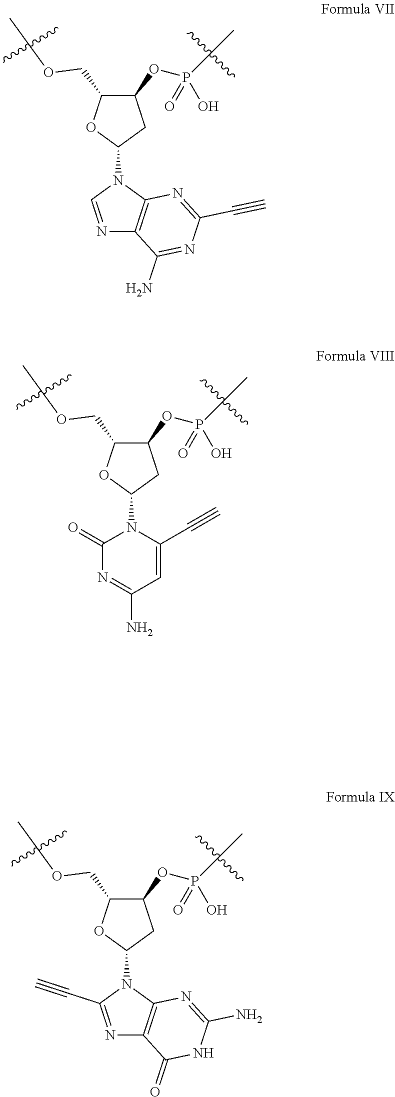

The Raman-active molecule in the oligonucleotide may comprise or consist of any one of Formulas II to IX described herein. The Raman-active molecule in the oligonucleotide may comprise or consist of any one of Formulas II to IX described herein, except wherein the alkyne group is substituted with an alternative Raman-active moiety, for example, selected from those described herein. The Raman-active molecule in the oligonucleotide may comprise or consist of any one of Formulas II to IX described herein, except wherein the alkyne group is located in a different position (preferably a position that does not affect the hybridisation capacity of the oligonucleotide), and optionally the alkyne group may be substituted with an alternative Raman-active moiety, for example, selected from those described herein. In one embodiment, the Raman-active molecule in the oligonucleotide comprises or consists of Formula II described herein. The Raman-active molecule in the oligonucleotide may comprise or consist of any one of Formula II described herein, except wherein the alkyne group is substituted with an alternative Raman-active moiety, for example, selected from those described herein. In one embodiment, the Raman-active molecule in the oligonucleotide comprises or consists of Formula III described herein. The Raman-active molecule in the oligonucleotide may comprise or consist of any one of Formula III described herein, except wherein the alkyne group is substituted with an alternative Raman-active moiety, for example, selected from those described herein.

In one embodiment, a plurality (i.e. two or more) of Raman-active moieties are provided in the genetic probe. For example, a single Raman-active molecule may be provided, which may have two or more Raman-active moieties incorporated therein. A single Raman-active molecule may be provided with one or more Raman-active moieties incorporated therein.

In one embodiment, the oligonucleotide of the genetic probe comprises a plurality (i.e. two or more) of Raman-active molecules, moieties or units (e.g. two or more base derivatives comprising a Raman-active moiety), for example for interrogating multiple target nucleotides in a target nucleic acid. The plurality of Raman-active molecules, moieties or units may be up to 5, 10, 15 or 20 Raman-active molecules, moieties or units. In another embodiment, the plurality of Raman-active molecules, moieties or units may be up to 20 Raman-active molecules, moieties or units. Each Raman-active molecule may have a different Raman-active moiety relative to each other.

The Oligonucleotide

The skilled person will recognise that the oligonucleotide may be of sufficient length to provide specific hybridisation with a complementary target nucleic acid (this may be complementary other than the specific point variation being targeted). The oligonucleotide may comprise 10 or more nucleotides. The oligonucleotide may comprise 15 or more nucleotides. For example, the oligonucleotide may consist of 10 to 1000 nucleotides, 10 to 500 nucleotides, 15 to 1000 nucleotides, 15 to 500 nucleotides, 10 to 60 nucleotides, or from 10 to 50, or from 10 to 40, or from 12 to 30, or from 15 to 25 nucleotides. In one embodiment, the oligonucleotide may be between about 10 and 90 nucleotides in length. In another embodiment, the oligonucleotide may be between about 10 and 100, or more, nucleotides in length. In another embodiment, the oligonucleotide may be at least about 12 nucleotides in length. In another embodiment, the oligonucleotide may be about 15 nucleotides in length. In another embodiment, the oligonucleotide may be no more than about 30 nucleotides in length. In another embodiment, the oligonucleotide may be no more than about 200 nucleotides in length. In another embodiment, the oligonucleotide may be no more than about 1000 nucleotides in length.

The oligonucleotide may in one embodiment be no more than about 150 nucleotides in length. In another embodiment, the oligonucleotide may be no more than about 100 nucleotides in length. In another embodiment, the oligonucleotide may be no more than about 90 nucleotides in length. In another embodiment, the oligonucleotide may be no more than about 40 nucleotides in length. In another embodiment, the oligonucleotide may be no more than about 30 nucleotides in length. In another embodiment, the oligonucleotide may be no more than about 20 nucleotides in length. In another embodiment, the oligonucleotide may be between about 10 and about 30 nucleotides in length.

Most preferably the oligonucleotide may be about a 20-mer strand, where preferably refers to an ideal scenario of synthesis efficiency (shorter) and specificity (longer). This offers the good chance to see a different between match and single mis-match upon hybridisation of the genetic probe and its target. There is a greater detectable difference upon hybridisation between a match and mis-match strand with the genetic probe if the modification is in the middle of a 20-mer strand, but this does not preclude incorporation of the Raman-active probe at any point along a sequence of any given length.

The oligonucleotide may comprise or consist of DNA. The oligonucleotide may comprise or consist of RNA. In one embodiment, the oligonucleotide is an oligoribonucleotide. In another embodiment, the oligonucleotide may comprise or consist of a nucleic acid analogue or derivative, such as a functional nucleic acid analogue or derivative having equivalent complementation as DNA or RNA. The oligonucleotide may comprise combinations of DNA, RNA and/or nucleotide analogues. Nucleic acid analogues may comprise PNA or LNA.

The Raman-active moiety, or the Raman-active molecule comprising the Raman-active moiety, may be located at any suitable position within the oligonucleotide, except it may not be positioned at an end of the oligonucleotide. The Raman-active moiety, or the Raman-active molecule comprising the Raman-active moiety, may be located at a position that will be opposing a nucleotide to be interrogated (e.g. a single point variant or a potential single point variant) when the oligonucleotide is hybridised/duplexed with the target nucleic acid. In one embodiment the Raman-active moiety, or the Raman-active molecule comprising the Raman-active moiety, is located substantially in the middle of the oligonucleotide (e.g. at or adjacent to nucleotide position 8 for a 15-mer, and for a further example at or adjacent to nucleotide positions 10 or 11 for a 20-mer, and for a further example at or adjacent to nucleotide positions 11 for a 21-mer). Adjacent is understood to be the immediate neighbouring nucleotide. Substantially in the middle of the oligonucleotide may be one of the middle 2, middle 3, middle 4, middle 5, or middle 6 bases.

The oligonucleotide may comprise a known/pre-determined sequence. The oligonucleotide may be complementary to the target nucleic acid sequence. The oligonucleotide may be 100% complementary to the target nucleic acid sequence, with the exception of the Raman-active moiety, or the Raman-active molecule comprising the Raman-active moiety, position. The oligonucleotide may be at least about 95%, or at least about 90% complementary to the target nucleic acid sequence. The oligonucleotide may be at least about 80% complementary to the target nucleic acid sequence. The oligonucleotide may be substantially complementary to the target nucleic acid sequence along the whole length of the oligonucleotide, with the exception of the base position comprising the Raman-active moiety (e.g. the position of the Raman-active molecule comprising the Raman-active moiety), which may be matched or mismatched, for example as per the design of the oligonucleotide. The oligonucleotide may be complementary to the target nucleic acid sequence along a length of at least about 8 consecutive nucleotides. The oligonucleotide may be complementary to the target nucleic acid sequence along a length of at least about 10 consecutive nucleotides. The oligonucleotide may be complementary to the target nucleic acid sequence along a length of at least about 14 consecutive nucleotides. The oligonucleotide may be complementary to the target nucleic acid sequence along a length of at least about 15 consecutive nucleotides. The oligonucleotide may be complementary to the target nucleic acid sequence along a length of at least about 20 consecutive nucleotides. The oligonucleotide may be sufficiently complementary to the target nucleic acid sequence to be able to selectively hybridise under stringent conditions. The oligonucleotide may hybridise to target nucleic acid, such as under stringent conditions. Indeed the Raman-active probe for single point variation may be matched to or with the sequence that displays the single point variance or include a mismatch to that single point variance, in either case the difference to between the matched and mismatched scenarios provides the diagnostic difference detected by Raman spectroscopy.

In one embodiment, the oligonucleotide comprises or consists of the P21 oligonucleotide: 5′-AGTCGCGXCTCAGCT-3′ (or a complementary sequence thereof), wherein X is the site of a nucleotide or nucleotide analogue (the Raman-active molecule) comprising the Raman-active moiety. In another embodiment, the oligonucleotide comprises or consists of the BRAF V600E oligonucleotide: 5′-AGATTTCXCTGTAGC-3′ (or a complementary sequence thereof), wherein X is the site of a nucleotide or nucleotide analogue (the Raman-active molecule) comprising the Raman-active moiety. In one embodiment, the oligonucleotide comprises or consists of the KRAS oligonucleotide: 5′-TACGCCAXCAGCTCC-3′ (or a complementary sequence thereof), wherein X is the site of a nucleotide or nucleotide analogue comprising the Raman-active moiety.

The oligonucleotide of the genetic probe may have a linear structure in the presence and/or absence of target nucleic acid hybridisation. The oligonucleotide of the genetic probe may not have a secondary structure, for example the oligonucleotide may not be arranged to form a hairpin loop structure. In one embodiment, the genetic probe does not require a conformational change in the oligonucleotide to act as genetic probe. In one embodiment, the binding of the genetic probe to its target does not result in a conformational change of the oligonucleotide.

In one embodiment, the genetic probe (or a plurality thereof) is free in solution (i.e. not anchored). In an alternative embodiment, the genetic probe (or a plurality thereof) is anchored or adsorbed to a surface. When anchored or adsorbed to a surface, the genetic probe, or groups of the genetic probe, may be spatially separated from other genetic probe(s) that are different, for example in an array. They may be different in sequence and/or have a different Raman-active molecule.

The skilled person will be familiar with a number of techniques and chemistries, such as click chemistry, to anchor or adsorb the oligonucleotide of the genetic probe to a surface.

The surface may be a planar surface, or the surface of a nanoparticle, such as a metal nanoparticle. In general, any appropriate chemistry may be used to anchor the oligonucleotide to the surface, for example click-chemistry may be used to anchor the oligonucleotide to the nanoparticle surface by reaction of a chemical group on the oligonucleotide with an opposing/complementary reactive group on the surface. The surface and/or the oligonucleotide may comprise reactive or charged groups for anchoring the oligonucleotide to the surface. The anchoring may be via use of a thiol anchor. In one embodiment a thiol anchor may attach to a thymine base on the oligonucleotide. The anchor may comprise a phosphoramidate bond. Alternatively, the anchor may comprise a triazole.

The oligonucleotide may be anchored by immobilisation using a carbodiimide crosslinker, such as EDC (also called EDAC; 1-Ethyl-3-(3-dimethylaminopropyl) carbodiimide hydrochloride, or DCC (dicyclohexyl carbodiimide). For example, the oligonucleotide may be anchored by immobilisation of using the carbodiimide linker upon a surface modified with stearic acid or octadecylamine. In another embodiment, the oligonucleotide may be anchored by immobilisation using a carbodiimide crosslinker, such as EDC, upon a surface modified with primary amino groups or aminoethanethiol. In another embodiment, the oligonucleotide may be anchored through attachment of nucleic acid, such as ssDNA, onto a phosphoric acid-terminated surface. The phosphoric acid may comprise MBPA (mercaptobutylphosphoric acid). In another embodiment, the oligonucleotide may be anchored through attachment of nucleic acid onto a film of aluminium alkenebisphosphonate on the surface. In another embodiment, the oligonucleotide may be anchored onto a mercaptosilane coating on the surface via the amino groups of the nucleic acid bases. In another embodiment, the oligonucleotide may be anchored using functionalised polypyrrole.

The nucleic acid may be anchored using any one of the covalent cross-linking reactions discussed in Pividori et al. Biosensors & Bioelectronics 15; pp. 191-303, 2000.

The oligonucleotide may comprise a modified nucleotide, comprising a reactive group to form an anchor. The reactive group for attachment to the surface may be termed an anchor unit. The oligonucleotide may comprise a modified thymine for use as an anchor. The anchor may comprise a modified thymine. The modified thymine may comprise a deoxythymidine (dT) modified with an anchor unit. The anchor unit may comprise thiol groups, such as dithiols. The anchor unit may comprise at least two or three dithiols as a surface anchor. The anchor unit may comprise a propagylamidopentanol linker attached to the thymine, such as at the C5 position of the thymine. In one embodiment, the oligonucleotide may comprise modified thymine comprising a deoxythymidine (dT) modified with anchor unit comprising three dithiols as a surface anchor and a propagylamidopentanol unit attached to the C5 position of the thymine. The reactive group to form an anchor may comprise biotin for linking with streptavidin, or comprise streptavidin for linking with biotin.

The oligonucleotide may be anchored to a modified surface by the use of silane coupling agents to introduce functional groups to the surface (such as thiols, amines, or aldehydes) for linking to a nucleic acid probe modified with an appropriate reactive group, which would form an anchoring bond.

It is known in the art to covalently attach oligonucleotides to surfaces through a thiol modification on the strand of DNA, forming a covalent bond, e.g. S—Au. See Sandstrom, P. et al, Langmuir 19, 7537-7543 (2003).

The most widely used method for covalently coating surfaces, such as nanoparticle surfaces, with thiol modified DNA is the ‘salt ageing’ method developed by the Mirkin group. See Hurst, S. J. et al, Anal. Chem. 78, 8313-8 (2006). The thiolated oligonucleotides are added to the surface(s)/nanoparticles in one addition, before the salt concentration is slowly increased. This increase in salt concentration is done over a period of many hours, as adding too much salt at once causes any citrate stabilised nanoparticles to aggregate. The salt allows maximum coating of the surface as it reduces the repulsion between the negatively charged oligonucleotides, allowing for closer packing on the surface.

Among alternative coating methods, Zhang et al. found that lowering the pH of the solution to 3.0 during the DNA attachment step allowed for rapid coverage of surfaces, such as nanoparticles surfaces. See Zhang, X. et al, J. Am. Chem. Soc. 7266-7269 (2012).

The technique of adding a thiol binding group to the oligonucleotide and subsequent attachment via the thiol group can be used in the present invention to anchor the oligonucleotide to the surface.

It is also known in the art to use a thioctic acid binding group. This has been found to be more stable, relative to the single thiol bond, when using gold surfaces. A bis-thiolated adduct is formed upon reduction of the disulphide which can bind to gold through both sulphur atoms. See Dougan, J. et al, Nucleic Acids Res. 35, 3668-75 (2007).

Thus an activated ester form of thioctic acid can be synthesised, e.g. as described in Stokes, R. J. et al, Chem. Commun. (Camb). 2811-2813 (2007). Meanwhile, an amine group can be added to the oligonucleotide backbone (e.g. the 5′ end), to provide an amine-terminated oligonucleotide. The activated ester can then be coupled to the amine-terminated oligonucleotide, by formation of an amide linkage, giving a thioctic acid modified oligonucleotide.

This technique of adding a thioctic acid binding group to the oligonucleotide and subsequent attachment via the thioctic acid binding group can be used in the present invention to anchor the oligonucleotide to the surface.

It will be appreciated, however, that the invention is not limited to attachment via a thioctic acid binding group.

In techniques where the oligonucleotide is modified to add a binding group, there will normally be a spacer group between the binding group and the oligonucleotide, sometimes referred to as the spacer region. It is known to vary this spacer group and its size. For example, it is known to use a polyethylene glycol (PEG) spacer group, and it has been found that this increases the loading of oligonucleotides onto the surface, when compared to a spacer group consisting of just 10 A bases or 10 T bases. See Hurst, S. J., et al, Anal. Chem. 78, 8313-8 (2006).

In the same reference it has also been described that sonication greatly increased the level of DNA coating on a surface. It was proposed that sonication could reduce the amount of non-specifically bound DNA, exposing more of the surface for the DNA to bind to.

Thus in the present invention it is possible to include a spacer group between the binding group and the oligonucleotide, e.g. a PEG spacer group, to assist with increasing the amount of oligonucleotide anchored to the surface.

Alternatively or additionally, in the present invention it is possible to use sonication to assist with increasing the amount of oligonucleotide anchored to the surface. For example, sonication for 10 seconds or more, or 15 seconds or more, e.g. from 20 to 60 seconds, may be used.

Another method known in the art for binding oligonucleotides to surface is to modify the surface with a highly cationic compound, such as quaternary ammonium chains. The negatively charged surface is then bound to the cationic nanoparticle through electrostatic interactions. This has been used by Sandhu et al. to successfully deliver DNA strands into cells with nanoparticles. See Sandhu, K. K. et al, Bioconjug. Chem. 13, 3-6 (2002).

This technique of attachment via surface modification of the nanoparticle to make it cationic, e.g. by functionalisation with quaternary ammonium chains, can be used in the present invention to anchor the oligonucleotide to the surface.

A benefit of having the oligonucleotide anchored to the surface is that in biological media and upon entering cells (e.g. when the surface is a nanoparticle surface), surface-immobilised DNA strands tend to be more resistant to nucleases that would otherwise quickly degrade the DNA. The genetic probes of the present invention are therefore useful for probing target species (e.g. mRNA) in biological environments.

The Single Targeted Nucleotide

The single targeted nucleotide may be a known or unknown nucleotide modification, such as a single point variation. In one embodiment, the nucleotide modification comprises or consists of a modification to a nucleotide known to cause cancer. In one embodiment, the nucleotide modification comprises or consists of a natural or synthetic modification.

Arrays

Two or more different genetic probes according to the invention may be provided in an array, for example in separate wells.

Therefore, according to another aspect of the present invention, there is provided an array of genetic probes, wherein the array of genetic probes comprises two or more genetic probes according to the invention provided in separate wells or on separate surfaces or spatially separated areas of the same surface.

The genetic probes may be compartmentalised relative to other different genetic probes (e.g. having an alternative nucleotide targeted).

The array may comprise two or more, or five or more, or ten or more, or fifty or more, or 100 or more, genetic probes according to the first aspect of the invention.

Multiple genetic probes in an array may be the same, or different to each other. For example, the oligonucleotide of one genetic probe may be arranged to hybridise to a different target nucleic acid relative to another genetic probe on the same surface. Additionally or alternatively the oligonucleotide of one genetic probe may be arranged to interrogate a different nucleotide position in the same target nucleic acid relative to another genetic probe on the same surface. This may be arranged by providing the Raman-active moiety, or Raman-active molecule comprising the Raman-active moiety, at different positions in the oligonucleotide backbone, or by modifying or shifting the sequence of the nucleotides flanking the Raman-active moiety, or Raman-active molecule comprising the Raman-active moiety. Additionally or alternatively the Raman-active moiety and/or Raman-active molecule, of a genetic probe may be different to the Raman-active moiety and/or Raman-active molecule, of the other genetic probe(s). For example, they may be arranged to resonate at different frequencies.

Method

According to another aspect of the present invention, there is provided a method of determining a single point variant nucleotide in a target nucleic acid in a pool of the target nucleic acid, the method comprising:
providing a genetic probe in accordance with the invention herein, wherein the genetic probe is capable of detecting a single point variant nucleotide,
wherein the genetic probe comprises an oligonucleotide that is substantially complimentary to the target nucleic acid,
and wherein the Raman-active moiety of the genetic probe is in a base position that is arranged to be paired with a nucleotide of the target nucleic acid to be interrogated;
contacting the genetic probe with the pool of target nucleic acid such that the genetic probe hybridises to the target nucleic acid;
measuring the Raman frequency shift generated by the duplex formation by Raman spectroscopy.

Advantageously, the method provides a rapid, cheap and reliable read-out out of the presence of single point variants. The DNA sequence can be targeted or, where appropriate, mRNA transcripts analysed indirectly (e.g. via cDNA formation and then PCR amplification) or directly (e.g. mRNA detection in cells) and in real time. Further advantageously, the method of the invention does not generate different read-outs through differences in target strand binding efficiency, rather through differences in the environment that it experiences upon duplex formation. This allows the sensing to be undertaken at any chosen fixed temperature (e.g. room or physiological temperature) so long as duplex formation occurs. The skilled person may adapt properties of the oligonucleotide including sequence length for optimisation of hybridisation at a given temperature.

According to another aspect of the present invention, there is provided a method of detecting a target nucleic acid in a pool of nucleic acid, the method comprising:
providing a genetic probe in accordance with the invention herein, wherein the genetic probe is capable of detecting the target nucleic acid,
wherein the genetic probe comprises an oligonucleotide that is substantially complimentary to the target nucleic acid,
and wherein the Raman-active moiety of the genetic probe is in a base position that is arranged to be paired with a nucleotide of the target nucleic acid to be interrogated;
contacting the genetic probe with the pool of nucleic acid such that the genetic probe hybridises to the target nucleic acid;
measuring the Raman frequency shift generated by the duplex formation by Raman spectroscopy.

The Raman-active moiety of the genetic probe may be incorporated into a nucleotide analogue, which is an analogue of A, T/U, C, or G. Such a nucleotide analogue may be matched or mis-matched with the target nucleotide of the target nucleic acid.

The Raman frequency shift generated upon duplex formation between the genetic probe and target nucleic acid may differ depending on the nucleotide's identity, such as a match or mis-match of a single point variation. The Raman frequency shift generated upon duplex formation between the genetic probe and target nucleic acid may be sufficient to distinguish between a match, a mis-match, and optionally a specific nucleotide A, T/U, C or G. In one embodiment, the Raman frequency shift for the target nucleic acid is compared to a known reference standard, and/or a known control molecule.

In one embodiment the reference standard is based on ambient nitrogen gas in air. In another embodiment the reference standard is based on nitrogen gas. In one embodiment, the Raman spectroscopy is carried out in a nitrogen atmosphere or an atmosphere comprising nitrogen, such as nitrogen in the ambient atmosphere (air). In one embodiment, N₂ gas is passed over the sample into the laser beam to act as a reference standard during the Raman spectroscopy. In one embodiment, the Raman mode of N₂ gas is used as an external calibration band. In one embodiment, the Raman mode of N₂ gas is used as an external calibration band and the Raman frequency shift is compared to a known value or measured Raman frequency of a matching nucleotide control. The N₂ gas may be in air.

The skilled person will recognise that for high resolution Raman measurements, especially when the Raman mode frequency of different sets of samples needs to be compared with high precision, the calibration is important. The main calibration requirements of Raman bands are their intensity, bandwidth, stability (thermal, temporal, etc.). Also, for high precision measurements the frequency of the calibrating Raman band needs to be close to the region (spectral window) of the measured band frequency in order to prevent errors from mechanical movements (of the grating). The Si band at 520 cm⁻¹, frequently used as a reference in the art, does not meet such criteria, especially for measurements of high frequency Raman modes. There is no known standard calibration Raman band in the region of alkyne stretching vibrations (˜2100 cm⁻¹). However, there is advantageously a band at 2329.6 cm⁻¹ characteristic of stretching vibration of N₂ molecules. The advantages of using this band as a reference standard for precise measurements of alkyne mode frequencies are as follows: the N₂ band is Raman active, narrow (˜5 cm⁻¹), stable in a wide temperature range, its frequency is located in one spectral window with the frequency of the alkyne band. Moreover, the use of this band is very easy and convenient because both pure nitrogenous gas and N₂ molecules from air can be used.

A mis-matched nucleotide of the target nucleic acid may be determined. In one embodiment, a mis-match between the genetic probe and the target nucleic acid generates a larger frequency shift than a match. A Raman frequency increase relative to a matched nucleotide may determine that the target nucleotide is a mismatch nucleotide. The Raman frequency shift may be at least +0.2 cm⁻¹ relative to a matched nucleotide Raman frequency. In another embodiment, the Raman frequency shift may be at least +0.5 cm⁻¹ relative to a matched nucleotide Raman frequency.

In one embodiment the temperature is maintained at fixed temperature in the range of about 4° C. to about 40° C. In another embodiment the temperature is maintained at fixed temperature in the range of about 20° C. to about 40° C. In another embodiment the temperature is at physiological temperature, such as about 37° C. The methods and use of the invention herein may be carried out at room temperature. Room temperature may be about 24° C., for example between about 20-26° C. The methods of the invention herein may be carried out below or substantially below the melting temperature of the oligonucleotide and the target nucleic acid, for example at least 5° C. the melting temperature of the oligonucleotide and the target nucleic acid. The methods of the invention herein may be carried out below 40° C., 35° C., 32° C., 30° C., or 28° C.

The target nucleic acid may be provided at concentrations of between 0.25 mM and 1 mM. In one embodiment, the target nucleic acid is provided at a concentration of at least 0.1 mM. In another embodiment, the target nucleic acid is provided at a concentration of at least 0.25 mM. In another embodiment, the target nucleic acid is provided at a concentration of at least 0.5 mM.

The pool of target nucleic acid may be in a sample. The sample may comprise a cell (e.g. whole cell), a cell lysate, a bodily fluid sample, or a nucleic acid sample, such as a sample of purified or partially purified nucleic acid. In one embodiment the sample comprises a cell or a population of cells, such as a eukaryote cell. The target nucleic acid may be eukaryote, prokaryote or viral nucleic acid. The eukaryote nucleic acid may be mammalian or fungal nucleic acid. In one embodiment the target nucleic acid is human. The target nucleic acid may be associated with a disease or condition or a known SNP. The target nucleic acid sequence may comprise or consist of DNA or RNA. The target nucleic acid sequence may comprise a mixture of DNA and RNA. The target nucleic acid sequence may comprise genomic nucleic acid. The target nucleic acid sequence may comprise viral RNA; mRNA; ncRNA; small RNA; and siRNA; or combinations thereof. The target nucleic acid sequence may comprise mitochondrial nucleic acid. The target nucleic acid sequence may comprise or consist of chromosomal and/or non-chromosomal DNA. In one embodiment, the target nucleic acid comprises circulating DNA, such as circulating tumour DNA (ctDNA)

In one embodiment, the target nucleic acid is associated with a disease or condition or a known single nucleotide variant.

In one embodiment, the target nucleic acid sequence comprises mRNA transcript. In another embodiment, the target nucleic acid sequence may comprise cDNA formed from mRNA transcripts. PCR amplification may be used to increase copy number prior to analysis, for example in the case of cDNA being detected.

In one embodiment, the target nucleic acid is a biomarker for a disease or condition. In one embodiment, the target nucleic acid is RNA associated with a concussion injury, or otherwise a suspected concussion injury. In particular, Pietro et al. (Br J Sports Med 2021; 0:1-10. doi:10.1136/bjsports-2020-103274), which is herein incorporated by reference, has noted the role of salivary small non-coding RNAs (sncRNAs) that can be used in the diagnosis of concussion, for example from sport-related concussion.

In one embodiment, the target nucleic acid is small non-coding RNA (otherwise know as microRNA), preferably salivary small non-coding RNA as described by Pietro et al. (Br J Sports Med 2021; 0:1-10. doi:10.1136/bjsports-2020-103274). In one embodiment, the target nucleic acid comprises one or more, or all of the small non-coding RNAs selected from let-7a-5p, miR-143-3p, miR-103a-3p, miR-34b-3p, RNU6-7, RNU6-45, Snora57, snoU13.120, tRNA18Arg-CCT, U6-168, U6-428, U6-1249, Uco22cjg1, and YRNA_255. In one embodiment, the target nucleic acid comprises let-7 and RNU6 family microRNAs. In a preferred embodiment, the target nucleic acid comprises Let-7f-5p. In another embodiment, the target nucleic acid comprises Let-7f-5p, and 1, 2, 3, 4, 5, 6, 7 or 8, or more nucleic acids selected from miR-143-3p, miR-103a-3p, miR-34b-3p, RNU6-7, RNU6-45, Snora57, snoU13.120, tRNA18Arg-CCT, U6-168, U6-428, U6-1249, Uco22cjg1, and YRNA_255.

The target nucleic acid may comprise one or more of let-7a-5p, let-7f-5p, miR-107, miR-148a-3p, miR-135b-5p, miR-21-5p, miR-34b-3p, miR-103a-3p and RNU6-45, which may be overexpressed in concussion, and miR-1246, which may be underexpressed in concussion.

In one embodiment, the target nucleic acid comprises one or more, or all of 7a-5p, miR-143-3p, miR-103a-3p, miR-34b-3p, RNU6-7, RNU6-45, Snora57, snoU13.120, tRNA18Arg-CCT, U6-168, U6-428, U6-1249, Uco22cjg1 and YRNA_255.

The cell or population of cells may be eukaryote or prokaryote. The cell or population of cells may be mammalian or fungal. The cell or population of cells may be human. The cell, population of cells, or sample may be derived from a patient. For example, it may be a patient having a condition, or suspected of having a condition, or at risk of having a condition. The cell, population of cells, or sample may be derived from a patient of unknown condition. The target nucleic acid, cell or population of cells may be from a subject who has, or is suspected to have, or is at risk of having, a condition associated with a single point variant. The single point variant in the target nucleic acid may be associated with a disease or condition. The single point variant in the target nucleic acid may be indicative of a disease or condition. The indication may be diagnostic or prognostic. The indication may be an indication of risk or likelihood of developing a disease or condition. Such conditions may comprise cancer or Alzheimer's Disease. In another embodiment, the condition may be Sickle Cell Anaemia. The skilled person will recognise that the invention herein would be useful for detecting and monitoring any disease or condition associated with a single point variant.

The condition or disease associated with a single point variant may comprise cancer, such as breast cancer, lung cancer, colorectal cancer, prostate cancer or melanoma. The lung cancer may be associated with a single point variant in the genes of PIK3CA, KRAS, NRAS, AKT1, ALK, or EGFR, or combinations thereof. The colorectal cancer may be associated with a single point variant in the genes of KRAS and/or PIK3CA. The breast cancer may be associated with a single point variant in BRAF. The prostate cancer may be associated with a single point variant in BRAF (BRAF 600E).

The condition associated with a SNP may comprise Alzheimer's disease or Sickle Cell Anaemia. The Alzheimer's Disease may be associated with a single point variant in the P21 gene.

The single point variant may in any of the genes selected from P21, BRAF, PIK3CA, KRAS, NRAS, AKT1, ALK, and EGFR, or combinations thereof.

The cancer may comprise cancer associated with a single point variant in the BRAF gene, such as some breast cancers and prostate cancers. The Alzheimer's Disease may be associated with an SNP in the P21 gene. The single point variant may comprise the P21 gene transversion (rs1801270; C to A); associated with Alzheimer's Disease. The single point variant may comprise BRAF gene transversion (V600E; X=T to A), which is associated with cancer.

The target nucleic acid may comprise sequence of the BRAF gene, or P21 gene. In one embodiment, the target nucleic acid comprises the P21 ribonucleic acid target: 3′-UCAGCGCXGAGUCGA-5′, wherein X is the site of the single point variant. In another embodiment, the target nucleic acid comprises the P21 deoxyribonucleic acid target: 3′-TCAGCGCXGAGTCGA-5′, wherein X is the site of the single point variant. In another embodiment, the target nucleic acid comprises the BRAF single point variant nucleic acid target: 3′-TCTAAAGXGACATCG-5′, wherein X is the site of the single point variant. In another embodiment, the target nucleic acid comprises the KRAS deoxyribonucleic acid target: 3′-GGA GCT GXT GGC GTA-5′, wherein X is the site of the single point variant.

The condition associated with a single point variant may comprise concussion.

In one embodiment, the single point variant comprises a sequence variation of a single nucleotide to an alternative nucleotide. The nucleotide that is subject to a variation/polymorphism may comprise adenine (A), thymine (T), cytosine (C), or guanine (G), or in the case of RNA, adenine (A), uracil (U), cytosine (C), or guanine (G).

According to another aspect of the present invention, there is provided the use of a genetic probe in accordance with the invention herein, for determining the single nucleotide identity of target nucleic acid in a pool of the target nucleic acid.

According to another aspect of the present invention, there is provided the use of a genetic probe in accordance with the invention herein, for diagnosis and/or prognosis of a condition associated with a single point variant in a subject.

According to another aspect of the present invention, there is provided the use of a genetic probe in accordance with the invention herein for detection of a target nucleic acid, such as microRNA, in a sample.

According to another aspect of the present invention, there is provided the use of a genetic probe in accordance with the invention herein, for diagnosis and/or prognosis of concussion in a subject that is associated with the presence of small non-coding RNA as described herein, for example in the saliva of the subject.

According to another aspect of the present invention, there is provided a kit for the detection of, and/or analysis of the ratio of, a single point variant of a target nucleic acid in a pool of the target nucleic acid, wherein the kit comprises:
the genetic probe according to the invention herein, wherein the Raman-active moiety is in a position that is arranged to be paired with a nucleotide of the target nucleic acid to be interrogated; and
optionally an internal reference standard to determine the probe concentration wherein the internal reference standard comprises a molecule of known concentration in the liquid.

According to another aspect of the present invention, there is provided a kit for the detection of, and/or analysis of target nucleic acid in a sample, wherein the kit comprises:
the genetic probe according to the invention herein, wherein the Raman-active moiety is in a position that is arranged to be paired with a nucleotide of the target nucleic acid to be interrogated; and
optionally an internal reference standard to determine the probe concentration wherein the internal reference standard comprises a molecule of known concentration in the liquid.

The internal reference standard may not affect and/or may not be involved in the hybridisation reaction. Preferably, the internal reference standard is arranged to have a detectable Raman peak within the recorded region of interest. The internal reference standard may comprise a Raman active molecule, for example a molecule comprising a Raman-active moiety, such as an alkyne group.

The kit may further comprise a spectrometer capable of Raman spectroscopy. Additionally, or alternatively, the kit may comprise nitrogen gas, for example provided in a cylinder.

The kit may comprise a buffer, such as sodium phosphate buffer. The kit may comprise an exonuclease, such as a T4 exonuclease to convert dsDNA to single stranded. The kit may further comprise primers and/or a polymerase for amplification, such as LAMP or PCR amplification, of the target nucleic acid. The primers may comprise Loop primers for loop mediated isothermal amplification (LAMP). The kit may further comprise a reverse transcriptase for conversion of RNA sequences to cDNA.

The sample may be a saliva sample from a subject. The sample, such as a saliva sample, may be taken within 48 hours of any suspected concussion. The sample, such as a saliva sample, may be taken within 36-48 hours of any suspected concussion. The sample, such as a saliva sample, may be taken within 24-48 hours of any suspected concussion.

Composition of Probes

According to another aspect of the invention, there is provided a composition comprising a plurality (e.g. two or more) of genetic probes according to the invention.

The composition may further comprise a solution, such as a buffer solution. The buffer solution may comprise sodium phosphate buffer.

Where reference is made to an oligonucleotide sequence, the skilled person will understand that one or more substitutions may be tolerated, optionally two substitutions may be tolerated in the sequence, such that it maintains the ability to hybridise to the target sequence, or where the substitution is in a target sequence, the ability to be recognized as the target sequence. References to sequence identity may be determined by BLAST sequence alignment (www.ncbi.nlm.nih.gov/BLAST/) using standard/default parameters. For example, the sequence may have at least 99% identity and still function according to the invention. In other embodiments, the sequence may have at least 98% identity and still function according to the invention. In another embodiment, the sequence may have at least 95% identity and still function according to the invention.

According to another aspect of the invention, there is provided a method of determining the status of a condition associated with a known single point variant in a subject, the method comprising:
providing a sample from the subject comprising a target nucleic acid, wherein the target nucleic acid may comprise the single point variant;
determining the presence or percentage of the single point variant in the sample relative to target nucleic acid not having the single point variant in accordance with the method of the invention herein,
wherein the presence or percentage of the single point variant is indicative of the status of the condition associated with the single point variant in the subject.

According to another aspect of the invention, there is provided a method of determining the status of a condition associated with a target nucleic acid in a subject, the method comprising:
providing a sample from the subject comprising, or potentially comprising, a target nucleic acid, wherein the target nucleic acid;
determining the presence or level of the target nucleic acid in accordance with the method of the invention herein,
wherein the presence or level of the target nucleic acid is indicative of the status of the condition associated with the target nucleic acid in the subject.

In one embodiment, the status may provide a diagnosis and/or prognosis for the condition. Additionally or alternatively, the status may comprise the progression of the condition. Further additionally or alternatively, the status may comprise the severity of the condition. The condition may be concussion, for example related to injury. The target nucleic acid may be a biomarker for a disease or condition. The target nucleic acid may comprise one or more of the microRNA described herein that are associated with concussion.

Other Aspects

According to another aspect of the present invention, there is provided a nucleic acid duplex, the nucleic acid duplex comprising the genetic probe according to the invention herein, duplexed with a target nucleic acid, wherein the Raman-active moiety incorporated within the genetic probe is base paired with a base of the target nucleic acid.

In one embodiment, the nucleic acid duplex does not have a secondary or tertiary structure. In one embodiment, the nucleic acid duplex does not have a hairpin loop structure.

The base of the target nucleic acid may be a single point variant, for example to be interrogated/detected.

According to another aspect of the invention, there is provided the use of a genetic probe in accordance with the invention herein, for determining the single point variant ratio or single nucleotide identity of target nucleic acid in a pool of the target nucleic acid.

The use may be in vitro. In another embodiment the use may be in vivo.

According to another aspect of the invention, there is provided the use of a genetic probe in accordance with the invention herein, for diagnosis and/or prognosis of a condition associated with a single point variant in a subject.

The condition or disease associated with a single point variant may comprise cancer, such as breast cancer, lung cancer, colorectal cancer or melanoma. The lung cancer may be associated with a single point variant in the genes of PIK3CA, KRAS, NRAS, AKT1, ALK, or EGFR, or combinations thereof. The colorectal cancer may be associated with a single point variant in the genes of KRAS and/or PIK3CA. The breast cancer may be associated with a single point variant in BRAF.

The condition associated with a single point variant may comprise Alzheimer's disease or Sickle Cell Anaemia. The Alzheimer's Disease may be associated with an single point variant in the P21 gene.

The single point variant may in any of the genes selected from P21, BRAF, PIK3CA, KRAS, NRAS, AKT1, ALK, and EGFR, or combinations thereof.

The condition associated with a single point variant may comprise Barrett's oesophagus or cancer, such as colorectal cancer. The colorectal cancer may be associated with MLH1 methylation. The single point variant may comprise methylation of MLH1.

According to another aspect of the present invention there is provided the use of N₂ gas to provide an external calibration band in a Raman spectroscopy assay.

According to another aspect of the present invention there is provided a method of Raman spectroscopy, wherein a Raman spectroscopy assay is carried out on a sample together with the use of N₂ gas to provide an external calibration band.

The term “genetic” in the context of genetic probe described herein is understood to mean a sensor or probe that is capable of analysis or interrogation of a nucleic acid sequence. Such term includes, but is not limited, to gene sequences, intergenic sequence, or any sequence of nucleic acid. Synthetic nucleic acid sequences may also be capable of analysis/interrogation.

The term “condition or disease associated with” used herein is understood to include a disease or condition of a subject that is directly or indirectly caused by the single point variant. The single point variant may or may not be the single causative modification leading to the condition or disease, for example the single point variant may contribute to the condition or disease in association with other contributing factors. The association may be a clinical association. The association may be a statistical association. The detection and/or finding of a particular ratio of a single point variant may indicate a higher risk of having or developing the disease or condition in a subject. Other modifications, symptoms or clinical manifestations may be used to contribute to determining the status, diagnosis or prognosis of the condition or disease.

The term “single point variant” used herein is understood to include any standard or non-standard variation to a given sequence, including a single nucleotide polymorphism (SNP), somatic mutations, single nucleotide modifications and mutations, such as a change in nucleotide base, or a modification of a base, such as methylation. The change or variation may be relative to wild-type, or relative to more prevalent bases or known sub-groups in a population, or relative to bases that are not associated with a disease or condition. In one embodiment, the single point variant may be relative to a standard/control sequence, such as a known sequence.

The term “comprising” is intended as encompassing all the specifically mentioned features as well optional, additional, unspecified ones, whereas the term “consisting of” only includes those features as specified. The term “comprising” may be substituted herein with the term “consisting”.

The term “base” is intended to refer to a nucleobase part of the system, sequence or molecule or an analogue thereof.

The term “oligonucleotide” may be understood by the skilled person as a molecule comprising oligonucleotide (polymer of nucleic acid), but other features and structures may also be provided. In another embodiment, the oligonucleotide may consist essentially of a polymer of nucleic acid. The term “oligonucleotide analogue” may refer to a polymer molecule that is analogous to a naturally occurring nucleic acid molecule, and which can function substantially the same as a nucleic acid molecule in terms of base pairing and hybridisation to a complementary nucleic acid. The skilled person will be aware of suitable oligonucleotide analogues, for example having bases linked with alternative backbone structures.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

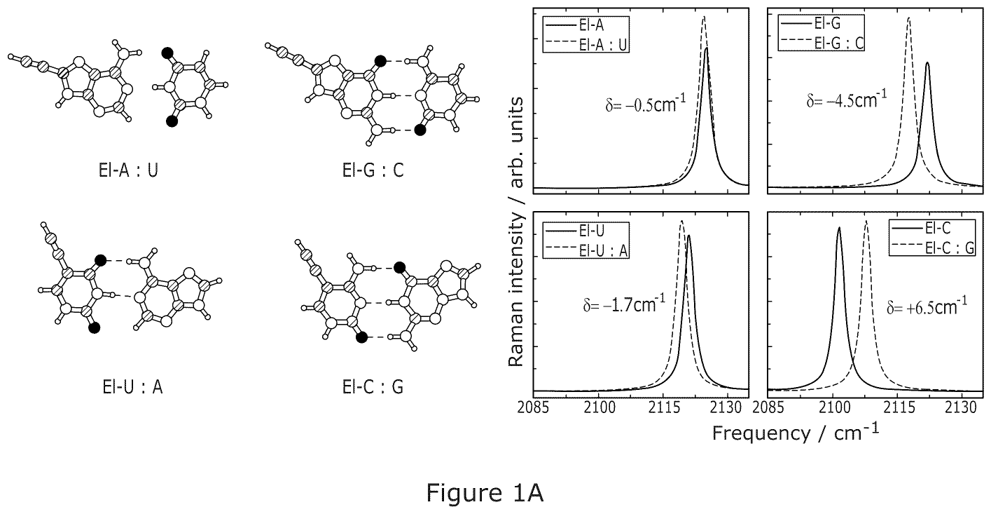

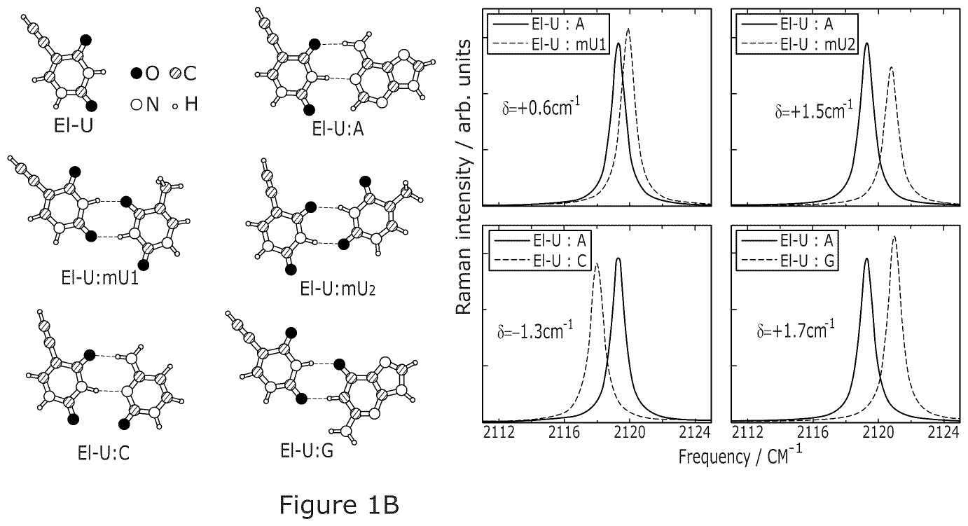

FIG. 1: Structure of nucleobase complexes (left) and simulated Raman spectra of ethynyl-labelled nucleobase molecules and their complexes in the region of —C≡C— vibration (right). (A, G, U and C denote adenine, guanine, uracil and cytosine molecules, respectively; El—ethynyl-labelled molecules. Calculated (B3LYP/6-311++G(df,pd)) Raman spectra of complexes were scaled by 0.9578. Lorenz-type function with full width at half maximum (FWHM) of 3 cm⁻¹ was used in order to simulate the spectra.

FIG. 1B: Structure of ethynyl uracil and nucleobase complexes (left) and simulated Raman spectra of different complexes in the region of —C≡C— vibration (right). (A, G, U, mU and C denote adenine, guanine, uracil, methyl-uracil and cytosine molecules, respectively; El—ethynyl-labelled molecule. Calculated (B3LYP/6-311++G(d,p)) Raman spectra of complexes were scaled by 0.95845. Lorenz-type function with full width at half maximum (FWHM) of 1 cm⁻¹ was used in order to simulate the spectra.

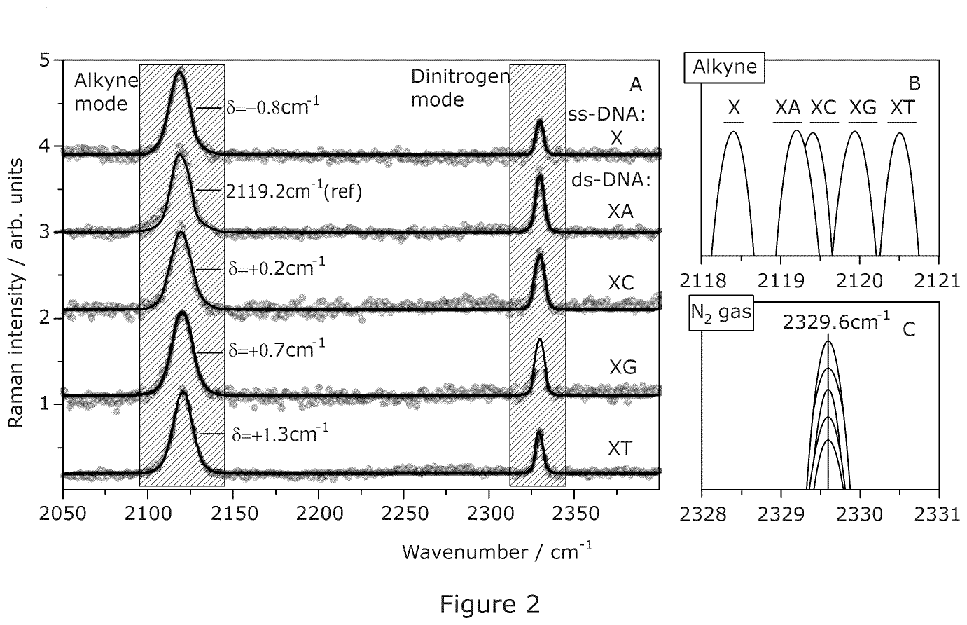

FIG. 2: (A) Baseline corrected and curve fitted Raman spectra of DNA sequences in the region of alkyne and N2 stretching vibrations, (B) variatmoions of alkyne Raman mode frequency in the Raman spectra of different samples and (C) the Raman mode of N2 gas used as external calibration band (X denotes EdU molecule).

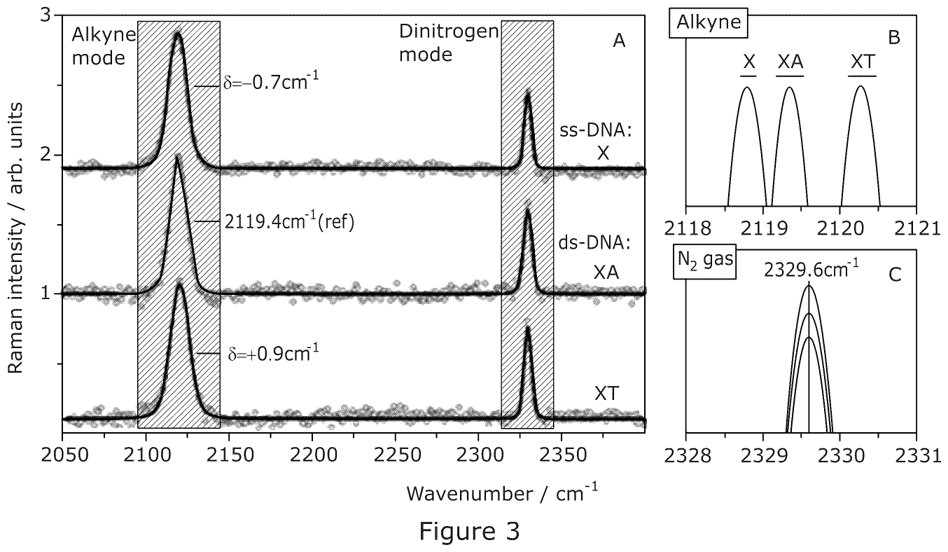

FIG. 3:(A) Baseline corrected and curve fitted Raman spectra of BRAF DNA sequences in the region of alkyne and N2 stretching vibrations, (B) variations of alkyne Raman mode frequency in the Raman spectra of different samples and (C) the Raman mode of N2 gas used as external calibration band (X denotes EdU molecule).

### EXAMPLES

Computer simulations were performed to calculate the Raman shift of the Watson-Crick base pairing compared with free Raman active molecules. The calculation was performed on the Hydrogen bonding of the nitrogenous part of the matching Watson-Crick base pairing molecules. The data (FIG. 1) suggested that the purine pairing C:G and G:C complexes were the most promising, demonstrating the larger frequency shifts (δ˜+6.5 and −4.5 cm⁻¹, respectively). The pyrimidine pairing El-U:A and A:U combination gave a predicted frequency shift of (˜−1.7 and −0.5 cm⁻¹). Due to the commercial availability of 5-Ethynyl-dU-CE Phosphoramidite, the El-U:A combination was chosen for our study. In this case the predicted frequency shift is small (˜−1.7 cm⁻¹) but it can still be resolved in the high resolution experimental Raman spectra.

Whilst the computed C:G and G:C complexes are most promising with larger frequency shifts (δ˜+6.5 and −4.5 cm⁻¹, respectively) between alkyne Raman modes of base pairs the clinical relevance of detecting T to A mutations meant the detection of A (by EI-U) was prioritized. In this case, the predicted frequency shift is small (˜−1.7 cm⁻¹) but it still can be resolved in the high-resolution experimental Raman spectra.

Inclusion of commercially available 5-ethynyl-dU-CE phosphoramidite in a probe sequence builds in the alkyne for Raman and is the natural base compliment for base A.

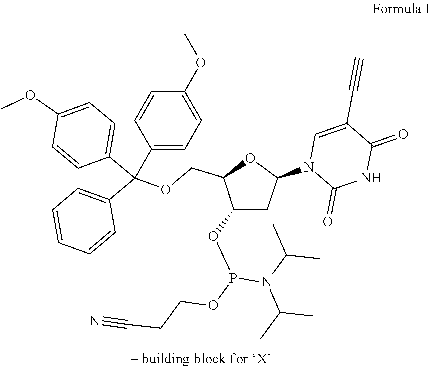 [SMILES: C#Cc1cn([C@H]2C[C@H](OP(OCCC#N)N(C(C)C)C(C)C)[C@@H](COC(c3ccccc3)(c3ccc(OC)cc3)c3ccc(OC)cc3)O2)c(=O)[nH]c1=O]

The material was purchased from Glen Research and used directly as instructed by the manufacturer on a solid support DNA synthesiser to make 15-mer strands. The probe and target strands were all made with the modification in the middle as highlighted in table 1. The probe strand had CXC where X is EdU and the target strands would be GXG, with X here being the four nucleobases (A, T, C, G). This would enable the measurement of the match and mis-match of all combinations and maybe reveal the identity of the base opposite the alkyne probe.

The reason for making a 15-mer strand is that, it offers the best chance to see a difference between match and single mis-match upon hybridisation of the probe and target. This is because there is a thermodynamic/detectable difference upon hybridisation between a match and mis-match strand with the probe if the modification is in the middle of a 15-mer strand. The test strand chosen has been studied by Tucker and co-workers in development of alternate signalling modalities for the proof of principle (POP) testing.

Sequences Made

TABLE 1
Oligo strands synthesised on automated
DNA synthesiser.

| | Oligonucleotide | |
|---|---|---|
| Strands | names | sequences |
| S0 | Natural probe | 5′-TGGACTCTCTCAATG-3′ |
| | 5′-CTC | |
| S1 | Probe 5′-CXC | 5′-TGGACTCXCTCAATG-3′ |
| S2 | Target 3′-GAG | 3′-ACCTGAGAGAGTTAC-5′ |
| S3 | Target 3′-GTG | 3′-ACCTGAGTGAGTTAC-5′ |
| S4 | Target 3′-GCG | 3′-ACCTGAGCGAGTTAC-5′ |
| S5 | Target 3′-GGG | 3′-ACCTGAGGGAGTTAC-5′ |
| S6 | Probe 5′-CXC | 5′-AGATTTCXCTGTAGC-3′ |
| | (BRAF) | |
| S7 | Target 3′-GAG | 3′-TCTAAAGAGACATCG-5′ |
| | (cancerous) | |
| S8 | Target 3′-GTG | 3′-TCTAAAGTGACATCG-5′ |
| | (wild type) | |

Sequence Synthetic Procedure:

Synthesis (Ultramild Reagents):

All nucleobases, reagents and solvents were purchased and used from suppliers without any further purification. Oligonucleotides were synthesised on an Applied Biosystems ABI 394 (Foster City, CA, 30 U.S.A). Standard phosphoramidites of Pac-dA, iPr-Pac-dG, Ac-dC, dT were purchased from LGC Genomics. 5-Ethynyl-dU-CE Phosphoramidite was purchase from Glen Research and used directly. The phosphoramidites were dissolved in anhydrous acetonitrile to a concentration of 0.1 M. Strands were synthesised at a 1 μmol scale on SynBase™ CPG 1000/110 solid supports from LGC Genomics.

The resins with the completed strands were placed in 1 ml solutions of potassium carbonate (0.05 M) in methanol and left overnight, before neutralisation with acetic acid (6 μl). Solvent was removed on a Thermo Scientific speed vac. The dried powders were re-dissolved in 1 ml ultrapure water and passed through a NAP-10 desalting column from GE Healthcare to remove any residual resin and potassium carbonate. The collected solutions were stored in the freezer before purification.

Purification and Characterisation:

Semi preparative HPLC purification was performed on an Agilent Technologies 1260 Infinity system using a Phenomenex Clarity 5 m Oligo-RP LC 250×10 mm column. Collected fractions were evaporated to dryness, redissolved in Milli-Q water (1 ml) and desalted using a NAP-10 column (GE Healthcare), whilst being eluted from the column to 1.5 ml. Purity was determined by analytical HPLC using a Phenomenex Clarity 5 m Oligo RP LC 250×4.6 mm column on an Agilent Technologies 1260 Infinity system. The UV/vis absorbance was monitored at 260 nm.

A solvent gradient system of HPLC grade acetonitrile (Fisher Scientific) and 0.1 M triethylamine acetate (TEAA) in HPLC grade water (Fisher Scientific) was employed for the purification of the oligonucleotides.

Pure oligonucleotides were characterised by negative mode electrospray mass spectrometry on a Waters Xevo G2-XS mass spectrometer. Sample concentrations were determined by optical density at 260 nm using a BioSpecNano micro-volume UV-Vis spectrophotometer from Shimadzu and the Beer Lambert law, with extinction coefficients obtained from Integrated DNA Technologies' OligoAnalyzer.

TM Result of Match and Mis-Match (POP Probe)

Having obtained the sequences, the stability/destabilisation effect of the modification upon duplex formation was determined by measuring the thermal melting (Tm) temperature. The result indicated that the modification did not seem to affect the stability of the strands upon hybridisation (comparing entries 1 and 3 in table 2). Although there was a slightly bigger destabilizing effect in the mis-match strand (comparing entries 2 and 4 in table 2). The numerical value was an approximately 10° C. difference between the match and mis-match in both the probe with modification and ones without. Attention was turned to a biologically relevant sequence that has also been prepared. The BRAF 600E is a single point variation for prostate cancer (T is wild-type. A denotes the cancerous mutant). Therefore, the ability to rapidly detect this SNIPs would be highly desired. The result in entry 5 and 6 indicate that there is a difference in the wild type (mis-match) and the cancerous (match) of around 10° C. This is in line with DNA stability of 15 mer strands observed previously. This indicates that the alkyne probe does not destabilise the parent structure and adds value to it being a non-intrusive probe.

TABLE 2

| entry | Duplex formations | Tm values |
|---|---|---|
| 1 | Natural strand- S0 & S2 | Match = |
| | (A match) | 64.0° C. |
| | 5′-TGGACTCTCTCAATG-3′ | |
| | 3′-ACCTGAGAGAGTTAC-5′ | |
| 2 | Natural strand- S0 & S3 | Mis-match = |
| | (T mis-match) | 54.5° C. |
| | 5′-TGGACTCTCTCAATG-3′ | |
| | 3′-ACCTGAGTGAGTTAC-5′ | |
| 3 | Modified strand- S1 & S2 | Match = |
| | (A match) | 64.7° C. |
| | 5′-TGGACTCXCTCAATG-3′ | |
| | 3′-ACCTGAGAGAGTTAC-5′ | |
| 4 | Modified strand- S1 & S3 | Mis-match = |
| | (T mis-match) | 53.7° C. |
| | 5′-TGGACTCXCTCAATG-3′ | |
| | 3′-ACCTGAGTGAGTTAC-5′ | |
| 5 | Modified strand- S6 & S7 | Match = |
| | (cancerous) | 62.3° C. |
| | 5′-AGATTTCXCTGTAGC-3′ | |
| | 3′-TCTAAAGAGACATCG-5′ | |
| 6 | Modified strand- S6 & S8 | Mis-match = |
| | (wild type) | 52.5° C. |
| | 5′-AGATTTCXCTGTAGC-3′ | |
| | 3′-TCTAAAGTGACATCG-5′ | |

Raman Spectroscopy

The accuracy required to reliably detect small changes in signal of the alkyne between free chain, chain bond to wild-type (healthy model—one mismatch) and mutated-type (disease model—no mismatches) required the development of a new spectroscopy technique.

Herein the first use of the Raman signal of ambient nitrogen gas in the air as a reference standard to achieve high resolution is disclosed.

Modelling has been performed to predict the EdU alkyne Raman peak shifts during perfectly matching (El-U:A) and mis-match (El-U:mU₁, El-U:mU₂, El-U:C, El-U:G) hybridizations. The modelling prediction (FIG. 1B.) was supported when Raman measurements were conducted (see table 3). The single strand probe (X=EdU) gave stretch at 2118.4 cm⁻¹. Upon hybridisation with the perfectly matching strand (EdU with A), there was a small shift to 2119.2 cm⁻¹. The result of the other strands containing one base mis-match (EdU with T, C or G) gave larger shifts (table 3). The mis-match of EdU with T gave the largest shift from 2118.4 cm⁻¹ to 2120.5 cm⁻¹. The extent of the shift is different for each mis-match base, suggesting that the identity of the mis-match base opposite the modification could be identified. While the value of the shift does not match the predicted value, the direction of the shifts is in good agreement with the calculated/predicted ones (except El-U:C) FIG. 1B. However, such calculations, and especially the geometry optimisation therein always have some limitations in case of systems consisting of a high number of atoms, whilst the experiments record the Raman response of the whole system. Therefore, the differences between predicted and measured should not be over interpreted. The fundamental result is that Raman Spectroscopy can be used to detect nucleoside hybridisation and single base mis-match.

TABLE 3
The shift in Raman signal from matched and
mis-matched strands.

| | | V | Av |
|---|---|---|---|
| Entries | DNA strands | (cm⁻¹) | (cm⁻¹) |
| 1 | Single strand- S1 | 2118.4 | −0.8 |
| | 5′-TGGACTCXCTCAATG-3′ | | |
| 2 | Duplex strand- S1 & S2 | 2119.2 | 0.0 |
| | (A match) | | (ref.) |
| | 5′-TGGACTCXCTCAATG-3′ | | |
| | 3′-ACCTGAGAGAGTTAC-5′ | | |
| 3 | Duplex strand- S1 & S3 | 2120.5 | +1.3 |
| | (T mis-match) | | |
| | 5′-TGGACTCXCTCAATG-3′ | | |
| | 3′-ACCTGAGTGAGTTAC-5′ | | |
| 4 | Duplex strand- S1 & S4 | 2119.4 | +0.2 |
| | (C mis-match) | | |
| | 5′-TGGACTCXCTCAATG-3′ | | |
| | 3′-ACCTGAGCGAGTTAC-5′ | | |
| 5 | Duplex strand- S1 & S5 | 2119.9 | +0.7 |
| | (G mis-match) | | |
| | 5′-TGGACTCXCTCAATG-3′ | | |
| | 3′-ACCTGAGGGAGTTAC-5′ | | |

(BRAF)

Attention was turned to a biologically relevant BRAF 600E sequence that has also been prepared. The single strand probe (S6, entry 1, table 4) has a peak at 2118.7 cm⁻¹; upon hybridisation with the matching strand (S7, A, cancerous mutant, entry 2, table 4), there was a small shift to 2119.4 cm⁻¹. With the mis-matching strand (T, wildtype, entry 3, table 4) the shift was larger, moving to 2120.3 cm⁻¹. The result here indicated that potentially Raman spectroscopy could be used to detect the BRAF 600E mutation SNIPs.

TABLE 4
Raman shift of BRAF sequence with matched and
mis-matched strands.

| | | v | Δv |
|---|---|---|---|
| Entries | BRAF 600E marker | (cm⁻¹) | (cm⁻¹) |
| 1 | Single strand- S6 | 2118.7 | -0.7 |
| | 5′-AGATTTCXCTGTAGC-3′ | | |
| 2 | Duplex strand- S6 & | 2119.4 | 0.0 |
| | S7 (A match) | | (ref.) |
| | 5′-AGATTTCXCTGTAGC-3′ | | |
| | 3′-TCTAAAGAGACATCG-5′ | | |
| 3 | Duplex strand- S6 & | 2120.3 | +0.9 |
| | S8 (T mis-match) | | |
| | 5′-AGATTTCXCTGTAGC-3′ | | |
| | 3′-TCTAAAGTGACATCG-5′ | | |

Concussion Associated RNAs:

| microRNAs | Gene Number |
|---|---|
| hsa-let-7f-5p | MIMAT0000067 |
| hsa-miR-1246 | MIMAT0005898 |
| hsa-miR-135b-5p | MIMAT0000758 |
| hsa-miR-21-5p | MIMAT0000076 |
| hsa-miR-425-5p | MIMAT0003393 |
| hsa-miR-497-5p | MIMAT0002820 |
| hsa-miR-148a-3p | MIMAT0000243 |
| hsa-let-7a-5p | MIMAT0000062 |
| hsa-let-7i-5p | MIMAT0000415 |
| hsa-miR-143-3p | MIMAT0000435 |
| hsa-miR-34b-3p | MIMAT0004676 |
| hsa-miR-144-3p | MIMAT0000436 |
| hsa-miR-16-1-3p | MIMAT0004489 |
| hsa-miR-103a-3p | MIMAT0000101 |
| hsa-miR-92a-3p | MIMAT0000092 |
| hsa-let-7b-5p | MIMAT0000063 |
| hsa-miR-142-5p | MIMAT0000433 |
| hsa-miR-29c-3p | MIMAT0000433 |
| has-miR-339-5p | MIMAT0000764 |
| has-miR-107 | MIMAT0000104 |
| hsa-miR-126-3p | MIMAT0000445 |
| hsa-miR-1271-5p | MIMAT0005796 |
| hsa-miR-143-3p | MIMAT0000435 |

| small | | | | | |
|---|---|---|---|---|---|
| non | | | | | |
| coding | | | | | |
| RNAS | Chr | Strand | Start | Stop | Sequence |
| RNU4-6p | X | − | 16893269 | 16893390 | TATCGTAGCCA |
| | | | | | ATGAGGTTTAT |
| | | | | | CCGAGGCGTGA |
| | | | | | TTATTGCTAAT |
| | | | | | TGAAAA |
| tRNA18 | 17 | + | 73030001 | 73030073 | GCACTGGCCTC |
| ArgCCT | | | | | CTAAGCCAGGG |
| | | | | | ATTGTGGGTTC |
| | | | | | GAGTCCCACCT |
| | | | | | GGGGTA |
| U6.428 | 1 | + | 180727858 | 180727953 | AAGATTAGCAT |
| | | | | | GAGGATGACAC |
| | | | | | GCAAATTCGTG |
| | | | | | AAGCGTTCCAT |
| | | | | | TTCTTT |
| RNU6-45 | 11 | + | 63737942 | 63738048 | GGCCCTTGTGC |
| | | | | | AAGGATGACAC |
| | | | | | GCAAATTCGTG |
| | | | | | AAGCGTTCCAT |
| | | | | | ATTTTT |
| RNU6-4 | 1 | − | 31970419 | 31970525 | GGCCCCTGCAC |
| | | | | | AGGGATGACAC |
| | | | | | GCAAATTCGTG |
| | | | | | AAGCGTTCCAT |
| | | | | | ATTTTT |
| RNU6-6 | 2 | + | 201694732 | 201694839 | GGCCCCTGTGC |
| | | | | | AAGGATGACAC |
| | | | | | GCAAATTCGTG |
| | | | | | AAGCGTTCCAT |
| | | | | | ATTTTT |
| RNU6-7 | 3 | + | 194935516 | 194935622 | GGCCCCTGCGC |
| | | | | | AAGGATGACAT |
| | | | | | GCAAATTCGTG |
| | | | | | AAGCGTTCCAT |
| | | | | | ATTTTT |
| RNU6-73 | 13 | + | 28402900 | 28403006 | GGCCCCTGTGC |
| | | | | | AAGGATGACAT |
| | | | | | GCAAATTCGTG |
| | | | | | AAGCGTTCCAT |
| | | | | | ATTTTT |
| SNORD3B | 17 | + | 18965225 | 18965440 | CTTCTCTCCGT |
| | | | | | TATTGGGGAGT |
| | | | | | GAGAGGGAGAG |
| | | | | | AACGCGGTCTG |
| | | | | | AGTGGT |
| tRNA120- | 6 | − | 28626014 | 28625085 | GCGCATGCTTA |
| AlaAGC | | | | | GCATGCATGAG |
| | | | | | GTCCCGGGTTC |
| | | | | | GATCCCCAGCA |
| | | | | | TCTCCA |
| tRNA73- | 6 | + | 28849165 | 28849237 | GCGTCTGATTC |
| ArgCCG | | | | | CGGATCAGAAG |
| | | | | | ATTGAGGGTTC |
| | | | | | GAGTCCCTTCG |
| | | | | | TGGTCG |
| U6.168 | 6 | − | 18307204 | 18307310 | ATGGCCCCTGC |
| | | | | | GCAAGGATGAC |
| | | | | | ACGCAAATTTG |
| | | | | | TGAAGGATTCC |
| | | | | | ATATTT |
| U6.375 | 4 | + | 109573306 | 109573412 | GGCCCCTGTGC |
| | | | | | AAGAATGACTC |
| | | | | | GCAAATTCGTG |
| | | | | | AAGCGTTCCAT |
| | | | | | ATTTTT |
| YRNA-684 | 18 | + | 20604559 | 20604666 | GCTTCTTTTAC |
| | | | | | TCTTTCCCTTC |
| | | | | | ATTCTCACTAC |
| | | | | | TGTACCTGATT |
| | | | | | CGTCTT |
| U6.601 | 19 | − | 39287642 | 39287749 | GGCCCCTGCGC |
| | | | | | AAGGATGACAT |
| | | | | | GCAAATTTGTG |
| | | | | | AAGTGTTCCAT |
| | | | | | ATTTTT |
| YRNA-255 | 17 | + | 80375102 | 80375197 | GUGUCACCAAC |
| | | | | | GUUGGUAUACA |
| | | | | | ACCCCCCACAA |
| | | | | | CUAAAUUUGAC |
| | | | | | UGGCUU |
| tRNA9- | 7 | + | 149255133 | 149255205 | CTTTTTGACTG |
| TyrGTA | | | | | TAGAGCAAGAG |
| | | | | | GTCCCTGGTTC |
| | | | | | AAATCCAGGTT |
| | | | | | CTCCCT |
| U2.3 | 1 | + | 150209315 | 150209504 | TCACTTCACGC |
| | | | | | ATCGATCTGGT |
| | | | | | ATTGCAGTACC |
| | | | | | TCCAGGAACAG |
| | | | | | TGCACC |
| U4.64 | 9 | + | 36267780 | 36267919 | GTATCGTAGCC |
| | | | | | AATGAGGTTTA |
| | | | | | TCCAAGGTGCG |
| | | | | | ATTATTGCTAA |
| | | | | | TTGAAA |
| SNORA57 | 10 | + | 27077946 | 27078086 | TGCTGGCGGCT |
| | | | | | TCCCATCCGCT |
| | | | | | GGTTCTATCCT |
| | | | | | CAAACGCCGGG |
| | | | | | ACACCG |
| UC022CJG1 | Y | + | 10037846 | 10037870 | CATTGATCATC |
| | | | | | GACACTTCGAA |
| | | | | | CGCACTTG |
| tRNA27- | 6 | + | 26766444 | 26765516 | GCGTCAGTCTC |
| MetCAT | | | | | ATAATCTGAAG |
| | | | | | GTCCTGAGTTC |
| | | | | | GAGCCTCAGAG |
| | | | | | AGGGCA |
| tRNA8- | 17 | + | 8090478 | 8090551 | GCGCCTGTCTA |
| ThrAGT | | | | | GTAAACAGGAG |
| | | | | | ATCCTGGGTTC |
| | | | | | GAATCCCAGCG |
| | | | | | GTGCCT |
| tRNA2- | 4 | − | 156384978 | 156385052 | GCATAAAACTT |
| LeuTAA | | | | | AAAATTTTATA |
| | | | | | ATCAGAGGTTC |
| | | | | | AACTCCTCTTC |
| | | | | | TTAACA |
| YRNA-245 | 2 | − | 25919945 | 25920057 | GTCTTTGTTGA |
| | | | | | ACTCTTTCCCT |
| | | | | | CCTTCTCATTA |
| | | | | | CTGTACTTGAC |
| | | | | | CAGTCT |
| snoU13.120 | 4 | + | 17530560 | 17530663 | GCTACCCTGGA |
| | | | | | ACCTTGTTATG |
| | | | | | ACATCTGCACA |
| | | | | | TTACCCATCTG |
| | | | | | ACCTGA |
| U6.1249 | 1 | + | 67661823 | 67661926 | GATGGCATGAC |
| | | | | | CCCTGATCAAG |
| | | | | | GACGGCATGCA |
| | | | | | AATTTGTGAAG |
| | | | | | TATTTC |
| tRNA84- | 1 | − | 1.61E+08 | 1.61E+08 | TTTCACCGCCG |
| GluTCC | | | | | CGGCCCGGGTT |
| | | | | | CGATTCCCGGT |
| | | | | | CAGGGAA |
| tRNA8- | 12 | + | 1.25E+08 | 1.25E+08 | TGCACGTATGA |
| AlaTGC | | | | | GGCCCCGGGTT |
| | | | | | CAATCCCCGGC |
| | | | | | ATCTCCA |

1. A genetic probe, wherein the genetic probe comprises:
an oligonucleotide, or an oligonucleotide analogue, with a Raman-active moiety incorporated therein, wherein the Raman-active moiety is incorporated into a base of the oligonucleotide or oligonucleotide thereof.

2. The genetic probe according to claim 1, wherein the genetic probe is suitable for determining the identity of a single targeted nucleotide in a target nucleic acid.

3. The genetic probe according to claim 1 or 2, wherein the Raman-active moiety comprises or consists of a moiety that resonates at a frequency within the cell-silent range of between 1800 and 2800 cm⁻¹.

4. The genetic probe according to any preceding claim, wherein the Raman-active moiety comprises or consists of one or more of the functional groups selected from a diyne group, an alkyne group, an azide group, a nitrile/cyano group, a metal-carbonyl complex, a carbon-13 label, and a deuterium group.

5. The genetic probe according to any preceding claim, wherein the Raman-active moiety comprises or consists of an alkyne group.

6. The genetic probe according to any preceding claim, wherein the Raman-active moiety is incorporated into a base to form a Raman-active molecule.

7. The genetic probe according to any preceding claim, comprising a Raman-active molecule in the oligonucleotide, wherein the Raman-active molecule comprises or consists of any one of Formulas II to IX, optionally wherein the alkyne group is substituted with an alternative Raman-active moiety.

8. The genetic probe according to any preceding claim, wherein the oligonucleotide comprises 10 or more nucleotides.

9. The genetic probe according to any preceding claim, wherein the oligonucleotide is a 20-mer strand.

10. The genetic probe according to any preceding claim, wherein the Raman-active moiety, or a Raman-active molecule comprising the Raman-active moiety, is located at a position that will be opposing a nucleotide to be interrogated when the oligonucleotide is hybridised/duplexed with the target nucleic acid.

11. The genetic probe according to any preceding claim, wherein the oligonucleotide comprises or consists of:
the P21 oligonucleotide: 5′-AGTCGCGXCTCAGCT-3′, or a complementary sequence thereof;
the BRAF V600E oligonucleotide: 5′-AGATTTCXCTGTAGC-3′, or a complementary sequence thereof;
the KRAS oligonucleotide: 5′-TACGCCAXCAGCTCC-3′, or a complementary sequence thereof;
wherein X is the site of a nucleotide or nucleotide analogue comprising the Raman-active moiety.

12. An array of genetic probes, wherein the array of genetic probes comprises two or more genetic probes according to any preceding claim provided in separate wells or on separate surfaces or spatially separated areas of the same surface.

13. A method of determining a single point variant nucleotide in a target nucleic acid in a pool of the target nucleic acid, the method comprising:
providing a genetic probe in accordance with any one of claims 1-11, wherein the genetic probe is capable of detecting a single point variant nucleotide,
wherein the genetic probe comprises an oligonucleotide that is substantially complimentary to the target nucleic acid,
and wherein the Raman-active moiety of the genetic probe is in a base position that is arranged to be paired with a nucleotide of the target nucleic acid to be interrogated;
contacting the genetic probe with the pool of target nucleic acid such that the genetic probe hybridises to the target nucleic acid;
measuring the Raman frequency shift generated by the duplex formation by Raman spectroscopy.

14. The method according to claim 13, wherein the Raman frequency shift for the target nucleic acid is compared to a known reference standard, and/or a known control molecule.

15. The method according to claim 14, wherein the reference standard is based on nitrogen gas to provide an external calibration band.

16. The method according to any of claims 13-15, wherein the pool of target nucleic acid is in a sample comprising a cell, a cell lysate, a bodily fluid sample, or a nucleic acid sample.

17. The method according to any of claims 13-16, wherein the target nucleic acid is associated with a disease or condition or a known single nucleotide variant.

18. Use of a genetic probe in accordance with any one of claims 1-11, for determining the single nucleotide identity of target nucleic acid in a pool of the target nucleic acid.

19. Use of a genetic probe in accordance with any one of claims 1-11, for diagnosis and/or prognosis of a condition associated with a single point variant in a subject.

20. A kit for the detection of, and/or analysis of the ratio of, a single point variant of a target nucleic acid in a pool of the target nucleic acid, wherein the kit comprises:
the genetic probe according to any one of claims 1-11, wherein the Raman-active moiety is in a position that is arranged to be paired with a nucleotide of the target nucleic acid to be interrogated; and
an internal reference standard to determine the probe concentration
wherein the internal reference standard comprises a molecule of known concentration in the liquid.

21. A composition comprising a plurality of genetic probes according to any one of claims 1-11.

22. A method of determining the status of a condition associated with a known single point variant in a subject, the method comprising:
providing a sample from the subject comprising a target nucleic acid, wherein the target nucleic acid may comprise the single point variant;
determining the presence or percentage of the single point variant in the sample relative to target nucleic acid not having the single point variant in accordance with the method of any of claims 12-17,
wherein the presence or percentage of the single point variant is indicative of the status of the condition associated with the single point variant in the subject.

23. Use of N₂ gas to provide an external calibration band in a Raman spectroscopy assay.

24. A method of Raman spectroscopy, wherein a Raman spectroscopy assay is carried out on a sample together with the use of N₂ gas to provide an external calibration band, optionally wherein the N₂ gas is ambient N₂ gas in air.

25. A method of detecting a target nucleic acid in a pool of nucleic acid, the method comprising:
providing a genetic probe in accordance with any one of claims 1-11, wherein the genetic probe is capable of detecting the target nucleic acid,
wherein the genetic probe comprises an oligonucleotide that is substantially complimentary to the target nucleic acid,
and wherein the Raman-active moiety of the genetic probe is in a base position that is arranged to be paired with a nucleotide of the target nucleic acid to be interrogated;
contacting the genetic probe with the pool of nucleic acid such that the genetic probe hybridises to the target nucleic acid;
measuring the Raman frequency shift generated by the duplex formation by Raman spectroscopy.

26. Use of a genetic probe in accordance with any one of claims 1-11, for detection of a target nucleic acid, such as microRNA, in a sample.

27. Use of a genetic probe in accordance with any one of claims 1-11, for diagnosis and/or prognosis of concussion in a subject that is associated with the presence of small non-coding RNA as described herein, for example in the saliva of the subject.

28. A kit for the detection of, and/or analysis of target nucleic acid in a sample, wherein the kit comprises:
the genetic probe in accordance with any one of claims 1-11, wherein the Raman-active moiety is in a position that is arranged to be paired with a nucleotide of the target nucleic acid to be interrogated; and
optionally an internal reference standard to determine the probe concentration wherein the internal reference standard comprises a molecule of known concentration in the liquid.

29. A method of determining the status of a condition associated with a target nucleic acid in a subject, the method comprising:
providing a sample from the subject comprising, or potentially comprising, a target nucleic acid, wherein the target nucleic acid;
determining the presence or level of the target nucleic acid in accordance with the method of any preceding claim,
wherein the presence or level of the target nucleic acid is indicative of the status of the condition associated with the target nucleic acid in the subject.

30. A nucleic acid duplex, the nucleic acid duplex comprising the genetic probe in accordance with any one of claims 1-11, duplexed with a target nucleic acid, wherein the Raman-active moiety incorporated within the genetic probe is base paired with a base of the target nucleic acid.
